(19) United States
(12) Patent Application Publication
(10) Pub. No.: US 2005/0169986 A1
(43) Pub. Date: Aug. 4, 2005

(54) Fast disintegrating tablets

(76) Inventors: Wei Tian, Minety (GB); Ann Leighton, Maidstone (GB); John Richard Langridge, Sint Oedenrode (NL)

Correspondence Address: MCANDREWS HELD & MALLOY, LTD, 500 WEST MADISON STREET, SUITE 3400, CHICAGO, IL, 60661, US

(21) Appl. No.: 10/505,916

(22) PCT Filed: Feb. 28, 2003
(86) PCT No.: PCT/GB03/00844

(30) Foreign Application Priority Data
Feb. 28, 2002 (GB) 0204771.0

(51) Int. Cl. A61K 9/20

(52) U.S. Cl. 424/464

(57) ABSTRACT

A fast disintegrating tablet comprising an active ingredient and one or more disintegrants characterised in that the tablet comprises agglomerates having an agglomerated particle size of at least 50 μm, said agglomerates comprising at least 10% by weight of a superdisintegrant selected from croscarmellose cellulose, crospovidone and sodium starch glycollate and being free of active ingredient.

This invention relates to fast disintegrating tablets and in particular to tablets which will disintegrate in the oral cavity within thirty seconds, preferably within fifteen seconds.

Fast dispersing solid dosage forms for oral administration are known. The dosage forms are particularly useful for patients who have difficulty in swallowing tablets e.g. children and elderly people.

Freeze drying processes have been used to prepare fast disintegrating dosage forms. Depending on the manufacturing process, the product obtained is characterised by a highly porous microstructure of the soluble supporting agent (i.e. mannitol, glycine, lactose, gelatins etc.) in which the active is homogeneously dispersed. Although this technology produces a product which rapidly disintegrates in water or in the oral cavity, a drawback is represented by the poor physical integrity of its physical structure which severely limits further manufacturing operations such as forming blister packs.

Another significant drawback of the freeze drying technology in manufacturing such dosage forms is the high production costs because of the lengthy duration of each freeze drying cycle (normally from 24 to 48 hours). The complexity of the industrial plants is another important factor which prejudices the large scale use of this technology for the development of rapid disintegrating tablets. Moreover, the thermal shocks, as a direct consequence of each freeze drying cycle, might physically modify the physical-chemical properties of the outer membrane of microencapsulated particles.

Rapid disintegrating tablets are known.

U.S. Pat. No. 6,106,861 discloses a rapidly disintergratable multiparticulate tablet which disintegrates in the mouth in less than forty seconds and which comprises an excipient and an active ingredient in the form of microcrystals coated with a coating agent. The excipient comprises, with respect to the mass of the tablet, from 3 to 15% by weight of at least one disintegration agent and from 40 to 90% by weight of at least one soluble diluent agent with binding properties consisting of a polyol having less than thirteen carbon atoms, said polyol being either in the directly compressible form which is composed of particles whose average diameter is from 100 to 500 micrometers or in the powder form which is composed of particles whose average diameter is less than 100 micrometers, said polyol being selected from the group consisting of mannitol, xylitol, sorbitol and maltitol, with the proviso that, when only one soluble diluent agent with binding properties is used, it is a polyol in the directly compressible form except sorbitol and, when at least two soluble diluent agents with binding properties are used, one is consisting of a polyol in the directly compressible form and the other is consisting of the same or another polyol in powder form, the proportion of directly compressible polyol to powder polyol being from 99/1 to 50/50.

U.S. Pat. No. 4,886,669 discloses a water-dispersible table comprising:

(a) microparticles which contain at least one pharmaceutically active substance

(b) at least one disintegrant and

(c) a swellable material which is able to generate a high viscosity when coming into contact with water and which is selected from the group consisting of guar gum, xanthan gum, alginates, dextran, pectins, polysaccharides, sodium or calcium carboxymethylcellulose, hydroxypropylcellulose and hydroxypropylmethylcellulose;

which tablet disintegrates rapidly in water forming a homogeneous suspension of high viscosity that can easily be swallowed.

WO99/44580 discloses a formulation for preparing a fast disintegrating tablet comprising a drug in multiparticulate form, one or more water insoluble inorganic excipients, one or more disintegrants; and optionally one or more substantially water soluble excipients, the amounts of said ingredients being such as to provide a disintegration time for the tablet in the mouth in the order of seventy-five seconds or less. It is stated superior tablet properties can be achieved by choosing appropriate amounts of the ingredients according to the classification shown below:

(A) insoluble ingredient; this includes the amount of drug either coated or uncoated and the amount of insoluble excipients including the insoluble inorganic salt used as filler/diluent, (e.g. di- or tri-basic calcium phosphate) organic filler (e.g. microcrystalline cellulose) or water insoluble lubricant (e.g. magnesium stearate, sodium stearyl fumarate, stearic acid or glyceryl behenate) and glidant (e.g. talc, silicon dioxide etc.)

(B) substantially soluble components e.g. the amount of compression sugars (e.g. lactose, flavouring agents, sweeteners (aspartame), binders (e.g. PVP) and surfactants etc.

(C) disintegrant, especially super-disintegrant such as maize starch or modified starches, cross-linked polyvinyl pyrrolidone or sodium carboxymethylcellulose.

For constant ratios of ingredients (A) and (B) increasing the amount of disintegrant generally gives poorer friability values and increased disintegration times. In view of this the amount of super disintegrant (C) should not be excessive and is therefore preferably in the range 0.5 to 30%, most preferably 1 to 20%, most preferably 2 to 15% by weight of the tablet.

WO00/09090 discloses an orally disintegrable tablet suitable for use in the delivery of at least one active ingredient in the form of microcapsules or powders characterised by between about 10 and about 80% of active ingredient containing microcapsules or powders by weight based on the weight of the tablet, said microcapsules or powder having a particle size ranging from between about 50 to 3,000 microns, an amount of at least one in-mouth viscosity enhancer, which is sufficient to provide a viscous, swallowable, organoleptically acceptable mass containing said microcapsules, within about three minutes when placed in a patient's mouth, said in-mouth viscosity enhancer being selected from the group consisting of methylcellulose, hydroxypropylmethyl cellulose, hydroxyethyl cellulose, carbopol and silicon dioxide; optionally between 0 and 60% of a rapidly dissolvable sugar or sugar alcohol filler by weight of the tablet selected from the group consisting of sucrose, mannitol, xylitol, lactose and maltose; optionally between 0 and about 35% of a binder by weight of the tablet selected from the group consisting of microcrystalline cellulose and methyl cellulose; optionally between 0 and about 40% of a disintegrant by weight based on the weight of the tablet selected from the group consisting of sodium starch glycolate and crospovidone; and optionally between 0 and 50% of an effervescent couple based on the weight of the tablet.

EP-A-914818 discloses a tablet comprising sugar alcohol or saccharide having an averaging particle diameter of not more than 30 μm, an active ingredient, and a disintegrant. In a preferred embodiment a wet granulation method using purified water, ethanol or the like is used to prepare the tablets in the method, for example, granulation can be executed by means of a general granulator such as a fluid-bed granulator, a rotary stirring granulator or an extruding granulator. The granulated material is dried, and mixed with a lubricant, and thereafter compressed into predetermined shape. Binder, sour agent, foaming agent, sweetening agent, flavouring agent, or colourant can be added as additive.

EP 1145711 discloses a flash-melt pharmaceutical dosage form comprising a medicament and a combination of four excipients consisting of a superdisintegrant, a dispersing agent, a distributing agent and a binder. The four excipients may be dry granulated with the medicament and suitable conventional ingredients.

EP 0281200 discloses a pharmaceutical tablet comprising an amphoteric β-lactam antibiotic, and as disintegrants, a cellulose product and low-substituted hydroxypropylcellulose, in which the cellulose product is microcrystalline or microfine cellulose or a mixture of both. The tablet may be formed by compressing a granule of β-lactam antibiotic and microcrystalline cellulose and/or microfine cellulose.

WO01/39746 discloses a method for improving the compressibility of a superdisintegrant, comprising causing a partial or complete internal co-transformation of superdisintegrant particles, comprising temporarily opening up said particles and adding an augmenting agent which enhances the properties of the superdisintegrant relative to the unmodified particles of the superdisintegrant. The superdisintegrant may be mixed with an active agent and compressed into solid dosage forms or may be subjected to a wet granulation with the active ingredient.

The possible ways of combining superdisintegrants into tablets may be classified as follows:
(1) blend—where all components of the tablet are mixed and compressed to form the tablet;
(2) extragranulation—where components of the tablet other than the superdisintegrant are granulated and the granules mixed with the superdisintegrant and compressed to form the tablet;
(3) intragranulation—where all components of the tablet are granulated and the granules compressed to form a tablet and
(4) granular disintegrant—in accordance with the invention where the superdisintegrant is granulated separately from the active ingredient and preferably alone or in combination with a water-soluble filler, and the granules of superdisintegrant mixed with the other tablet components (which may be granular) and compressed to form a tablet.

Therefore according to the present invention there is provided a fast disintegrating tablet comprising an active ingredient and one or more disintegrants characterised in that the tablet comprises agglomerates having an agglomerated particle size of at least 50 μm, said agglomerates comprising at least 10% by weight of a superdisintegrant selected from croscarmellose cellulose, crospovidone and sodium starch glycollate and being free of active ingredient.

The invention also provides a method of making a fast disintegrating tablet comprising the steps of

(i) forming agglomerates having an average agglomerated particle size of at least 50 μm and comprising one or more superdisintegrants selected from croscarmellose cellulose, crospovidone and sodium starch glycollate such that the agglomerates comprise at least 10% by weight of superdisintegrant and the agglomerates are free of active ingredient

(ii) mixing the agglomerates from step (I) with an active ingredient and optionally other tableting excipients, and

(iii) compressing the mixture from step (ii) to form a tablet.

It has been surprisingly found that the disintegration time of tablets may be significantly reduced when a superdisintegrant is agglomerated prior to its incorporation into tabletting mixtures.

The agglomeration of the disintegrant improves disintegration time in a simple and effective manner. Tablets according to the invention may have a smooth surface, pleasing mouthfeel that is free of grittiness and disintegrate within thirty seconds, preferably within fifteen seconds according to the standard European Pharmacopoeia disintegration test.

It is preferred that all of the disintegrant is present in the form of agglomerates. However, disintegrant may be present in non-agglomerated form provided that at least 50%, preferably at least 75%, more preferably at least 90% by weight of disintegrant is agglomerated.

The agglomerates comprise at least 10%, preferably at least 25%, generally from 25 to 100% by weight disintegrant. The remainder of the agglomerates may comprise known tabletting ingredients including water-soluble and water insoluble fillers and/or diluents, binder, flavouring agents etc. Preferably the agglomerates comprise from 25 to 100% by weight disintegrant, the remainder being a water-soluble filler and optionally a binder, such as citric acid.

The active ingredients can include pharmaceutical ingredients, vitamins, minerals and dietary supplements. Pharmaceutical ingredients may include, without limitation, antacids, analgesics, anti-inflammatories, antipyretics antibiotics, antimicrobials, laxatives, anorexics, antihistamines, antiasthmatics, antidiuretics, anti-flatuents, antimigraine agents, biologicals (proteins, peptides, oligonueleotides, etc.) anti-spasmodics, sedatives, antihyperactives, antihypertensives, tranquillisers, decongestants, beta blockers and combinations thereof.

The amount of active ingredient used can vary greatly. The size of the dosage form, the requirements of other ingredients, and the number of, for example, tablets which constitute a single dose will all impact the upper limit on the amount of pharmacologically active ingredient which can be used. However, generally, the active ingredient is provided in an amount of up to about 80% by weight of the finished tablet and, more preferably, in a range of between greater than 0 and about 60% by weight thereof. For example, the active ingredient can be included in an amount of between 1 microgram to about 2 grams, and more preferably between about 0.01 and about 1000 milligrams per tablet.

The active ingredient may be in the form of a powder having a particle size of 50 to 3000 μm, generally 100 to 2000 μm. The active ingredient may also be in the form of microcapsules having a similar size range. The microcapsules may have an enteric, sustained release or targeted release coating. By sustained release it is understood that while the microcapsules are rapidly dispersed in the mouth the active ingredients or drug itself is released from the microcapsules slowly or in a manner that alters its otherwise normal release profile. By the use of these coatings, the time necessary between doses of drug can be extended relative to the use of the same quantity of uncoated particles or microcapsules. The extended release coatings may provide for a release of drug, with as uniform a rate as possible, over a period of time ranging from between four to forty-eight hours and usually from between four to twenty-four hours. The targeted release coating may facilitate the release of actives at a pre-determined site along the gastrointestinal tract e.g. ileum, duodenum, jenenum or colon.

The active ingredient can be taste modified or masked by any means known in the art. This can include the use of intense sweeteners, favours, flavouring agents, taste modifiers and taste inhibitors, the application of a delayed release coating and the incorporation of the active into a micromatrix formed by an interpenetrating polymer network.

Disintegrating agents suitable for use in the present formulations include pharmaceutical excipients which facilitate the break-up of a tablet when it is placed in aqueous environment. Disintegrants once in contact with water, swell, hydrate, change in volume or form to produce a disruptive force that opposes the efficiency of the binder(s) causing the compressed tablet to break apart. They belong to different morphological classes and possess different functionality properties. The agglomerates used in the invention comprise a superdisintegrant selected from:

(1) sodium starch glycolate which is available as PRIMOJEL® and EXPLOTAB® and EXPLOSOL.

(2) crospovidones available as e.g. POLYPLASDONE XL® and KOLLIDON XL®.

(3) croscarmellose cellulose as, e.g., AC-DI-SOL®, PRIMELLOSE®, PHARMACEL XL®, EXPLOCEL® and NYMCEL ZSX®.

Other disintegrants may also be present e.g. alginic acid and sodium alginate, microcrystalline cellulose, e.g. AVICEL®, PHARMACEL®, EMCOCELL® and VIVAPUR® and methacrylic acid-divinylbenzene copolymer salts available as e.g. AMBERLITE® IRP-88.

Substantially water-soluble components that may be used in the present invention include sugars or soluble fillers, e.g. lactose, sucrose, dextrose, mannitol, etc., flavouring agents, sweeteners e.g. aspartame, saccharine etc., pH adjusting agents e.g. fumaric acid, citric acid, sodium acetate etc., binders e.g. polyethylene glycols, soluble hydroxyalkylcellulose, polyvinylpyrrolidone, gelatins, natural gums etc., surfactants e.g. sorbitan esters, docusate sodium, sodium lauryl sulphate, cetrimide etc., soluble inorganic salts e.g. sodium carbonate, sodium bicarbonate, sodium chloride etc.

Substantially water insoluble inorganic excipients include for example, water insoluble fillers and/or diluents, e.g. salts such as dibasic calcium phosphate, calcium phospate tribasic, calcium sulfate and dicalcium sulfate. Advantageously the particle size of the water insoluble inorganic excipient is such that at least 35% of the particles are larger than 75 μm. Most preferably at least 80% of the particles are larger than 75 μm.

The amount of superdisintegrant is generally at least 2% by weight of the tablet and preferably at least 4% by weight; a useful range being 4 to 20% by weight. Increasing levels of disintegrant tend to give poorer friability values for the tablet.

The amount of water-soluble and water-insoluble materials may be selected over wide ranges, depending upon the desired properties of the tablet.

The agglomeration of the superdisintegrant may be accomplished by any means known in the art, for example, wet granulation, dry granulation, extrusion, spray drying, co-spray drying, spray agglomeration etc. The average particle size of the agglomerator is at least 50 μm. Increasing particle size decreases disintegration time. Particle size ranges of from 75 to 500 μm are useful. Larger particle sizes may adversely affect the appearance of the tablets.

Tablets according to the present invention can be manufactured by well known tableting procedures. In common tableting processes, the agglomerates and other materials are deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion. Various tableting methods, well known to those skilled in the art, are comprehensively discussed throughout Pharmaceutical Dosage Forms: Tablets, Second Edition, edited by Herbert A. Lieberman et al., Copyright 1989 by Marcel Dekker, Inc., as well as other well known texts.

The invention will be illustrated by the following Examples in which the following ingredients were used:
Polyplasdone® XL-10: crospovidone having an average particle size of about 30 μm
Mannitol: mannitol having an average particle size of about 60 μm
Explotab®: sodium starch glycolate having an average particle size about 40 μm

All parts and percentages are by weight unless otherwise stated.

### Test Procedure: Determination of Disintegration Time According to European Pharmacopoeia (PhEur) Method

The test apparatus consists of six plastic tubes (each has an internal diameter of 28 mm) and a disk of rust-proof wire gauze fitted at the lower end of the tubes (so as to form a basket). The basket is suspended in a glass beaker containing purified water at a temperature between 36 and 38° C.

Tablets are placed in the tubes (one tablet per tube), which are raised and lowered repeatedly in a uniform manner. The disintegration time is determined as the time between when the basket containing tablets is lowered into the water and when there are no particles remaining above the gauze.

### EXAMPLE 1 (INVENTION)

50 parts of cross-linked povidone, Polyplasdone® XL-10 and 50 parts of mannitol were thoroughly mixed and then agglomerated with purified water in a Kenwood Magimix 4100 food processor. The agglomerates were dried in a forced air oven at 55° C. and screened through a 500 micron sieve.

Powdered mannitol was separately agglomerated with purified water in a Diosna granulator and dried in a Niro fluid bed drier at 60° C.

The cross-linked povidone agglomerates (13 parts) were then blended in with 86 parts of agglomerated mannitol and 1 part of magnesium stearate to form a tablet mixture. Tabletting was carried out using magnesium stearate to form a tablet mixture. Tabletting was carried out using a Manesty® F3 press and 10 mm normal concave punches. The tablets weighted 310 grams, had a hardness of 1.5 kp and an EP disintegration time of 10.5 seconds.

### EXAMPLE 1a (COMPARATIVE)

A tablet mixture was prepared by dry blending 6.5 parts of Polyplasdone® XL-10, 92.5 parts of agglomerated mannitol and 1 part of magnesium stearate. The mixture was tabletted by a Manesty® F3 press using 10 mm normal concave punches. The tablets weighed 314 grams, had a hardness of 1.4 kp and an EP disintegration time of 22 seconds.

### EXAMPLE 1b (COMPARATIVE)

6.5 parts of Polyplasdone® XL-10 and 92.5 parts of mannitol were thoroughly mixed and then agglomerated with purified water as in Example 1. The agglomerates were dried in a forced air oven at 55° C. A tablet mixture was prepared by dry blending 99 parts of the agglomerates and 1 part of magnesium stearate. Tabletting was carried out using a Manesty® F3 press and 10 mm normal concave punches. The tablets weighed 311 grams, had a hardness of 1.7 kp and an EP disintegration time of 28 seconds.

These Examples demonstrate that the invention provides tablets having improved disintegration compared to tablets found by compression of a dry blend (Example 1a) or after agglomerating all the sugar alcohol and disintegrant as disclosed in EP-A-914818.

### EXAMPLES 2, 2a, 2b

The same procedures were followed as in Examples 1, 1a and 1b except that sodium starch glycolate (Explotab®) was used instead of cross-linked povidone.

The tablets had the following characteristics:

| | Weight (mg) | Hardness (kp) | EP DT (s) |
|---|---|---|---|
| Example 2 | 346 | 1.6 | 15 |
| Example 2a | 369 | 1.2 | 30 |
| Example 2b | 318 | 1.5 | 36 |

These Examples demonstrate the improved disintegration time obtained with tablets of the invention.

### EXAMPLE 3

The same procedure was followed as in Example 1 except that different amounts of agglomerates were incorporated into tablets. The agglomerates used were as in Example 1, 50% cross-linked povidone and 50% mannitol. The amounts of agglomerates used and tablet properties are reported in the following Table.

| | | % Mg | Tablet weight | Tablet hardness |
|---|---|---|---|---|
| % Agglomerate | % Mannitol | Stearate | (mg) | (kp) |
| 4 | 95 | 1 | 309 | 1.6 |
| 8 | 91 | 1 | 302 | 1.6 |
| 12 | 87 | 1 | 302 | 1.4 |
| 16 | 83 | 1 | 308 | 1.7 |

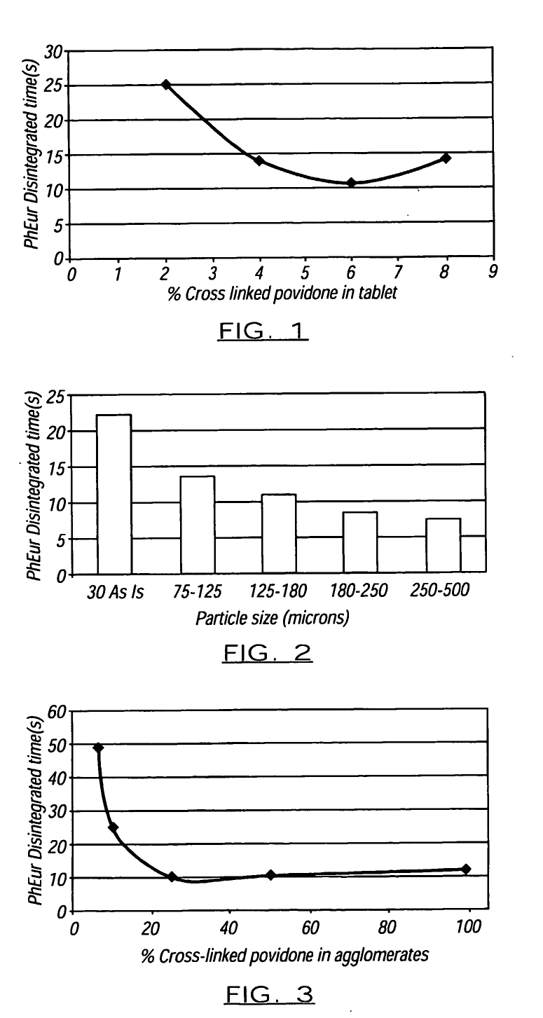

The effect of cross-linked povidone concentration in tablets on disintegration time is shown in FIG. 1.

### EXAMPLE 4

The same procedure was followed as in Example 1 except that the disintegrant agglomerates contained 100% cross-linked povidone and the agglomerates were fractionated to different particle size fractions by sieving. All tablets contained 6.5% cross-linked povidone agglomerates, 1.0% magnesium stearate and 92.5% mannitol. The particle sizes and properties of the tablets are reported in the following Table. The effect of agglomerate size on disintegration is illustrated in FIG. 2.

| | Tablet | Hardness |
|---|---|---|
| | weight (mg) | (kp) |
| 30 μm (non-agglomerated) | 314 | 1.4 |
| 75 to 125 μm | 301 | 1.4 |
| 125 to 180 μm | 325 | 1.7 |
| 180 to 250 μm | 320 | 1.8 |
| 250 to 500 μm | 314 | 1.8 |

### EXAMPLE 5

The same procedure was followed as Example 1 except that the disintegrant agglomerates contained different amounts of cross-linked povidone.

However, the final concentration of cross-linked povidone in tablets were maintained at 6%. The following Table reports the weight ratio of cross-linked povidone to mannitol, the concentration of cross-linked povidone agglomerate and agglomerated mannitol and the tablet properties. 1% magnesium stearate was added as lubricant. The agglomerates used had a particle size of 125 to 250 μm.

| Agglomerate | | | | |
|---|---|---|---|---|
| composition | % Agglomerate | | Tablet weight | Hardness |
| Pov:Man* | in tablet | % Mannitol | (mg) | (kp) |
| 100:0 | 6.0 | 92.5 | 320 | 1.7 |
| 50:50 | 12 | 87 | 302 | 1.4 |
| 25:75 | 24 | 75 | 300 | 1.7 |
| 10:90 | 60 | 38 | 325 | 1.4 |
| 6.5:93.5 | 92.5 | 6.5 | 286 | 1.7 |

*cross-linked povidone:mannitol weight ratio

The effect of cross-linked povidone concentration in the agglomerates on disintegration time is shown in FIG. 3.

### EXAMPLE 6

The same procedure was followed as Example 1 except that the disintegrant agglomerates contained two parts of cross-linked povidone and one part of mannitol. The disintegrant agglomerates were sieved and the size fraction between 75 μm and 500 μm collected. Tablets incorporating disintegrant agglomerates and unmodified disintegrant (as is) having the compositions reported in the following Table with a hardness ranging from 0.9 kp to 2.7 kp were prepared using a Manesty F3 press.

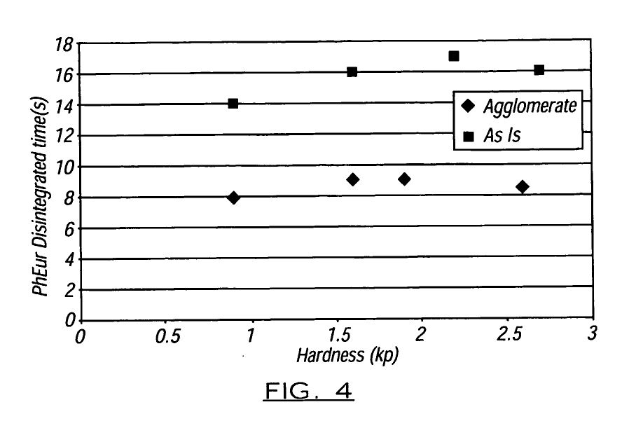

Disintegration time was determined as described previously and shown in the following FIG. 4 of the accompanying drawings.

| | Control (as is) | Invention |
|---|---|---|
| Mannitol | 93 | 90 |
| Cross-linked povidone (as is) | 6 | — |
| Disintegrant agglomerate (2 | — | 9 |
| parts cross-linked povidone and | | |
| 1 part mannitol) | | |
| Magnesium stearate | 1 | 1 |

### In the Following Examples the Following Test Procedure was Used to Determine Oral Disintegration Time

Disintegration of the tablet was carried out by placing one tablet on the tongue of the subject. The subject was instructed not to bite the tablet but allowed to move the tablet gently within the mouth. The disintegration time was determined as the time between the tablet was placed in the mouth and when the last noticeable granules were disintegrated.

### EXAMPLE 7

Formulation of sildenafil tablet:

| Formulation component | mg/tablet |
|---|---|
| Sildenafil granules | 185.75 |
| Agglomerated disintegrant granules | 60.00 |
| Aspartame | 1.25 |
| Lemon flavour | 0.50 |
| Magnesium stearate | 2.50 |
| Total | 250.0 |

Sildenafil granules were prepared according to the following formulation:

| Formulation component | % |
|---|---|
| Sildenafil citrate | 37.81 |
| Mannitol SD200 | 45.19 |
| Vivastar (sodium starch glycolate) | 7.00 |
| Citric acid | 5.00 |
| Lactitol | 5.00 |
| Total | 100.00 |

To prepare the sildenafil granule, citric acid and lactitol were dissolved in water. Sildenafil citrate, mannitol SD200 and sodium starch glycolate were blended in a food processor for 10 minutes, the citric acid/lactitol solution was added and mixed in, and the resulting wet granules were dried in a tray drier at 50° C.

Agglomerated disintegrant granules were prepared according to the following formulation:

| Formulation component | % |
|---|---|
| Mannitol (M60) | 60.00 |
| Polyplasdone XL-10 | 25.00 |
| Citric acid | 7.50 |
| Lactitol | 7.50 |
| Total | 100.00 |

To prepare the agglomerated disintegrant granules, citric acid and lactitol were dissolved in deionised water, mannitol and polyplasdone were dry mixed for 10 minutes in a food mixer. The citric acid/lactitol solution was added to the dry mixture to form wet granules. The wet granules were dried in a forced air oven at 50° C. to achieve a moisture level of less than 2%. The dried granules were screened and the 75 to 250 micron size range was obtained.

Tableting: the sildenafil granules and agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Stoke B2 rotary press fitted with 16 stations of ⅜ inch (9.525 mm) normal concave tooling.

The tablets had an average weight of 252 mg and a mean crushing strength of 1.1 kp. The oral disintegration time was 28 seconds.

### EXAMPLE 8

Tablets incorporating concentrated sildenafil granules.

Formulation of sildenafil tablet:

| Formulation component | mg/tablet |
|---|---|
| Sildenafil granules | 76.77 |
| Mannitol granules | 107.73 |
| Agglomerated disintegrant granules | 60.00 |
| Aspartame | 2.00 |
| Lemon flavour | 1.00 |
| Magnesium stearate | 2.50 |
| Total | 250.0 |

Sildenafil granules were prepared according to the following formulation:

| Formulation component | % |
|---|---|
| Sildenafil citrate | 91.50 |
| Lemon flavour | 1.00 |
| Aspartame | 2.50 |
| Citric acid | 2.50 |
| Lactitol | 2.50 |
| Total | 100.00 |

To prepare the sildenafil granule, citric acid and lactitol were dissolved in water. Sildenafil citrate, lemon flavour and aspartame were blended in a food processor for 10 minutes, the citric acid/lactitol solution was added and mixed in, and the resulting wet granules were dried in a tray drier at 50° C.

Mannitol granules were prepared according to the following formulation.

| Formulation component | % |
|---|---|
| Mannitol (SD200) | 91.00 |
| Vivastar (sodium starch glycolate) | 4.00 |
| Citric acid | 2.50 |
| Lactitol | 2.50 |
| Total | 100.00 |

To prepare the mannitol granules, citric acid and lactitol were dissolved in deionised water, mannitol and Vivastar were mixed for 10 minutes in a food mixer. The citric acid/lactitol solution was added to the dry mixture to form wet granules. The wet granules were dried in a forced air oven at 50° C. to achieve a moisture level of less than 1%.

Agglomerated disintegrant granules were prepared according to Example 7.

Tableting: the sildenafil granules, mannitol granules, agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 252.5 mg and a mean crushing strength of 1.1 kp. The oral disintegration time was 12 seconds.

### EXAMPLE 9

Tablets incorporating concentrated sildenafil granules and an increased amount of sweetener.

Formulation of sildenafil tablet:

| Formulation component | mg/tablet |
|---|---|
| Sildenafil granules | 90.00 |
| Mannitol granules | 95.00 |
| Agglomerated disintegrant granules | 60.00 |
| Lemon flavour | 2.50 |
| Magnesium stearate | 2.50 |
| Total | 250.0 |

Sildenafil granules were prepared according to the following formulation:

| Formulation component | % |
|---|---|
| Sildenafil citrate | 78.04 |
| Acesulfame K (high intensity | 16.40 |
| sweetener) | |
| Citric acid | 2.78 |
| Lactitol | 2.78 |
| Total | 100.00 |

To prepare the sildenafil granules, citric acid and lactitol were dissolved in water. Sildenafil citrate and acesulfame K were blended in a food processor for 10 minutes, the citric acid/lactitol solution was added and mixed in, and the resulting wet granules were dried in a tray drier at 50° C.

Mannitol granules were prepared according to Example 8.

Agglomerated disintegrant granules were prepared according to Example 7.

Tableting: the sildenafil granules, mannitol granules, agglomerated disintegrant granules were placed in a suitable container. Lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 251.1 mg and a mean crushing strength of 1.4 kp. The oral disintegration time was 15 seconds.

### EXAMPLE 10

Tablets incorporating concentrated sildenafil granules and a solubilisation inhibitor.

Formulation of Sildenafil Tablet:

| Formulation component | mg/tablet |
|---|---|
| Sildenafil granules | 116.00 |
| Mannitol granules | 58.50 |
| Agglomerated disintegrant granules | 60.00 |
| Lemon flavour | 5.00 |
| Acesulfame K | 8.00 |
| Magnesium stearate | 2.50 |
| Total | 250.0 |

Sildenafil Granules were Prepared According to the Following Formulation:

| Formulation component | % |
|---|---|
| Sildenafil citrate | 63.70 |
| Acesulfame K | 8.71 |
| Sodium carbonate | 27.59 |
| Total | 100.00 |

To prepare the sildenafil granule, sildenafil citrate, sodium carbonate and acesulfame K were blended in a food processor for 10 minutes, distilled water was added was added and mixed in, and the resulting wet granules were dried in a tray drier at 50° C.

Mannitol granules were prepared according to Example 8.

Agglomerated disintegrant granules were prepared according to Example 7.

Tableting: the sildenafil granules, mannitol granules, agglomerated disintegrant granules were placed in a suitable container. Acesulfame K and lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

The tablets had an average weight of 260.0 mg and a mean hardness of 0.9 kp. The oral disintegration time was 10 seconds.

1. A fast disintegrating tablet comprising an active ingredient and one or more disintegrants characterised in that the tablet comprises agglomerates having an agglomerated particle size of at least 50 μm, said agglomerates comprising at least 10% by weight of a superdisintegrant selected from croscarmellose cellulose, crospovidone and sodium starch glycollate and being free of active ingredient.

2. A fast disintegrating tablet as claimed in claim 1 in which said agglomerates consist of the superdisintegrant and optionally one or more excipients selected from water-soluble and water-insoluble fillers and diluents, binders and flavouring agents.

3. A fast disintegrating tablet as claimed in claim 2 in which said agglomerates consist of superdisintegrant and water-soluble filler and optionally binder.

4. A fast disintegrating tablet as claimed in claim 1 in which the agglomerates comprise at least 25% by weight of superdisintegrant.

5. A fast disintegrating tablet as claimed in claim 4 in which the agglomerates comprise from 25 to 100% by weight of superdisintegrant.

6. A fast disintegrating tablet as claimed in claim 1 in which at least 50% of the disintegrant is present in the tablet is in the form of said agglomerates.

7. A fast disintegrating tablet as claimed in claim 6 in which all of the disintegrant in the tablet is present in the form of agglomerates.

8. A fast disintegrating tablet as claimed in claim 1 in which the average particle size of the agglomerates is in the range 75 to 500 μm.

9. A fast disintegrating tablet as claimed in claim 1 in which the tablet comprises at least 2% by weight of superdisintegrant.

10. A fast disintegrating tablet as claimed in claim 9 in which the tablet comprises from 4 to 20% by weight of superdisintegrant.

11. A fast disintegrating tablet as claimed in claim 1 in which the superdisintegrant is a combination of cross-linked povidone and sodium starch glycolate.

12. A fast disintegrating tablet as claimed in claim 1 in which the tablet additionally comprises water-soluble fillers or diluents.

13. A fast disintegrating tablet as claimed in claim 12 in which the water-soluble fillers or diluents are selected from lactose, sucrose, dextrose and mannitol.

14. A fast disintegrating tablet as claimed in claim 1 in which the tablet additionally comprises water-insoluble excipients.

15. A fast disintegrating tablet as claimed in claim 1 in which the active ingredient is present in the form of a powder having a particle size of from 50 to 3000 μm is in the form of microcapsules or in a micro-matrix formed by an interpenetrating polymer network.

16. A fast disintegrating tablet as claimed in claim 15 in which the microcapsule or micro-matrix has a taste masked, enteric, sustained or targeted release coating.

17. A fast disintegrating tablet as claimed in claim 1 further comprises a taste modifying agent.

18. A method of making a fast disintegrating tablet comprising the steps of
(i) forming agglomerates having an average agglomerated particle size of at least 50 μm and comprising one or more superdisintegrants selected from croscarmellose cellulose, crospovidone and sodium starch glycollate such that the agglomerates comprise at least 10% by weight of superdisintegrant and the agglomerates are free of active ingredient,
(ii) mixing the agglomerates from step (I) with an active ingredient and optionally other tableting excipients, and
(iii) compressing the mixture from step (ii) to form a tablet.

19. A method of making a fast disintegrating tablet as claimed in claim 18 in which the ingredients of the mixture which is compressed comprise one or more water soluble fillers or diuents.

20. A method of making agglomerates for a fast disintegrating tablet comprising the step of forming agglomerates having an average agglomerated particle size of at least 50 μm and comprising one or more superdisintegrants selected from croscarmellose cellulose, crospovidone and sodium starch glycollate such that the agglomerates comprise at least 10% by weight disintegrant and are free of active ingredient.
